(19) United States
(12) Patent Application Publication
(10) Pub. No.: US 2025/0256049 A1
(43) Pub. Date: Aug. 14, 2025

(54) RADON INHALATION DEVICE

(71) Applicant: NIHON HAKO SANGYO COMPANY LIMITED, Tokyo (JP)

(72) Inventor: Jyunichi SASAKI, Tokyo (JP)

(73) Assignee: NIHON HAKO SANGYO COMPANY LIMITED, Tokyo (JP)

(21) Appl. No.: 18/857,243

(22) PCT Filed: Feb. 24, 2023
(86) PCT No.: PCT/JP2023/006833

§ 371 Date: Oct. 16, 2024

(30) Foreign Application Priority Data
Apr. 20, 2022 (JP) 2022-069137

(51) Int. Cl. A61M 16/10; A61M 16/12

(52) U.S. Cl. CPC A61M 16/108; A61M 16/105; A61M 16/125; A61M 2202/0208; A61M 2205/36

(57) ABSTRACT

It is possible for users to inhale radon at a stable concentration regardless of the surrounding atmospheric temperature to gain effects similar to those of radon hot springs. The radon inhalation device 1 includes a radon generation container 5 and a heater 6 that heats the inside of the radon generation container 5. The radon generation container 5 includes a container main body 4 the inside of which is divided into a radon reservoir 8 and an external air opening chamber 10 by the bag 11, radioactive material containing ores 7 stored inside the radon reservoir 8, and a radon extractor 14 that communicates with the inside of the radon reservoir 8 and the outside of the container main body 4. External air can be introduced when the bag 11 is deformed to increase the volume of the radon reservoir 8, and the radon inside the radon reservoir 8 is released outside the container main body 4 when the gas inside an internal space 39 is suctioned from the outside of the container main body 4.

### TECHNICAL FIELD

The present invention relates to a radon inhalation device that allows users to efficiently inhale radon, which offers similar effects with those of a radon hot spring cure.

### BACKGROUND ART

Conventionally in Japan, therapeutic effects on various diseases of Miasa hot springs and Tamagawa hot springs, famous as radon hot springs, have been widely known. In Austria and Germany, radon inhalation therapy is recognized as a medical treatment for nerve pain, peripheral circulatory disturbance, and the like. Various devices have been proposed in light of the advantageous effects of hot springs containing radiation decay products such as radon.

For example, in a radon inhalation device that has been known, ores containing radioactive materials are stored in a bottomed cylindrical container sealed at its opening with a lid equipped with an air supply tube through which external air is taken into the container, and users can inhale radon emitted from the radioactive ores inside the container with the inhalation tube attached to the lid (see Patent Document 1).

### CITATION LIST

### Patent Literature

Patent Document 1: Japanese Unexamined Patent Application Publication No. 2007-313274

### SUMMARY OF INVENTION

### Technical Problem

Such a conventional radon inhalation device has a problem that users cannot inhale radon at a desired concentration (a concentration required to gain therapeutic effects similar to those of radon hot springs) when it is used in an environment having low surrounding atmospheric temperature.

Therefore, the purpose of the present invention is to provide a radon inhalation device that allows users to inhale radon at a stable concentration regardless of the surrounding atmospheric temperature to gain effects similar to those of radon hot spring.

### Solution to Problem

A radon inhalation device according to the present invention comprising: a radon generation container for producing radon; and a heater that heats an inside of the radon generation container,
wherein the radon generation container includes: a container main body an inside of which is divided into a radon reservoir and an external air opening chamber by a bag; a radioactive material containing ore stored inside the radon reservoir; and a radon extractor that communicates with an inside of the radon reservoir and an outside of the container main body,
wherein when the bag is deformed to increase a volume of the radon reservoir, the radon extractor can introduce external air to the radon reservoir, and when gas in an internal space is suctioned from the outside of the container main body, the radon extractor releases radon inside the radon reservoir to the outside of the container main body.

### Advantageous Effects of Invention

According to the present invention, the inside of the radon generation container can be heated with a heater to a temperature appropriate for radon production, users can inhale radon at a stable concentration regardless of the surrounding atmospheric temperature to gain effects similar to those of radon hot spring.

### BRIEF DESCRIPTION OF DRAWINGS

FIG. 1 is an external perspective view of a hot spring cure box storing therein a radon inhalation device according to a first embodiment of the present invention.

FIG. 2A is a diagram showing a radon inhalation device according to the first embodiment of the present invention, a plan view of a radon inhalation device 1.

FIG. 2B is a diagram showing the radon inhalation device according to the first embodiment of the present invention, a plan view of the radon inhalation device 1.

FIG. 2C is a diagram showing the radon inhalation device according to the first embodiment of the present invention, a right lateral view showing a container main body 4 of the radon inhalation device 1 partially cut out.

FIG. 2D is a diagram showing the radon inhalation device according to the first embodiment of the present invention, an enlarged view of a part of the radon inhalation device 1 shown in FIG. 2C.

FIG. 3A is a diagram showing a radon extractor used in the radon inhalation device, a plan view of a radon extractor 14.

FIG. 3B is a diagram showing the radon extractor used in the radon inhalation device, a frontal view of the radon extractor 14.

FIG. 3C is a diagram showing the radon extractor used in the radon inhalation device, a right lateral view of the radon extractor 14.

FIG. 3D is a diagram showing the radon extractor used in the radon inhalation device, an enlarged cross-sectional view of the radon extractor 14 of FIG. 3B cut out along a line A1-A1.

FIG. 4A is an exploded view of the radon extractor.

FIG. 4B is an exploded view of the radon extractor.

FIG. 4C is an exploded view of the radon extractor.

FIG. 4D is an exploded view of the radon extractor.

FIG. 4E is an exploded view of the radon extractor.

FIG. 5A shows a first use state of the radon inhalation device, which is a state of use of the radon inhalation device 1 before radon inhalation.

FIG. 5B shows the first use state of the radon inhalation device, which is a state of use of the radon inhalation device 1 during radon inhalation.

FIG. 6 shows a second use state of the radon inhalation device.

FIG. 7A shows a third use state of the radon inhalation device, which is a state of use of the radon inhalation device 1 before introduction of external air.

FIG. 7B shows the third use state of the radon inhalation device, which is a state of use of the radon inhalation device 1 during introduction of external air.

FIG. 8 is a fourth use state of the radon inhalation device.

FIG. 9A shows a state of a bag storing radioactive containing ores in a powdered form to be placed in the radon inhalation device, and is a plan view thereof.

FIG. 9B shows a state of a bag storing radioactive containing ores in a powdered form to be placed in the radon inhalation device, and is a cross-sectional view of the bag cut out along a line A2-A2 of FIG. 9A.

FIG. 10 is a diagram explaining a variation of the radon inhalation device.

FIG. 11 shows a variation of the first embodiment.

FIG. 12 is a diagram showing the radon inhalation device according to the second embodiment of the present invention, and shows the container main body partially cut out.

FIG. 13A shows the sixth embodiment of the radon inhalation device.

FIG. 13B is the sixth embodiment of the radon inhalation device.

FIG. 14 is a cross-sectional view of the bag sandwiched between a lower case member and an upper case member.

FIG. 15 is a plan view of the lower casing member from above.

FIG. 16 shows a configuration of a gas channel.

FIG. 17 shows a configuration of an external air introduction channel.

FIG. 18 shows a configuration of an air suction channel.

FIG. 19A shows the radon inhalation device installed in a cabinet.

FIG. 19B shows a diagram of the radon inhalation device installed in the cabinet.

FIG. 20A shows a diagram of a case which is almost entirely filled with a radon reservoir 8.

FIG. 20B shows a diagram of the case which is almost entirely filled with an external air opening chamber 10.

### DESCRIPTION OF EMBODIMENTS

Hereinafter, embodiments of the present invention are described in detail with reference to the drawings.

### First Embodiment

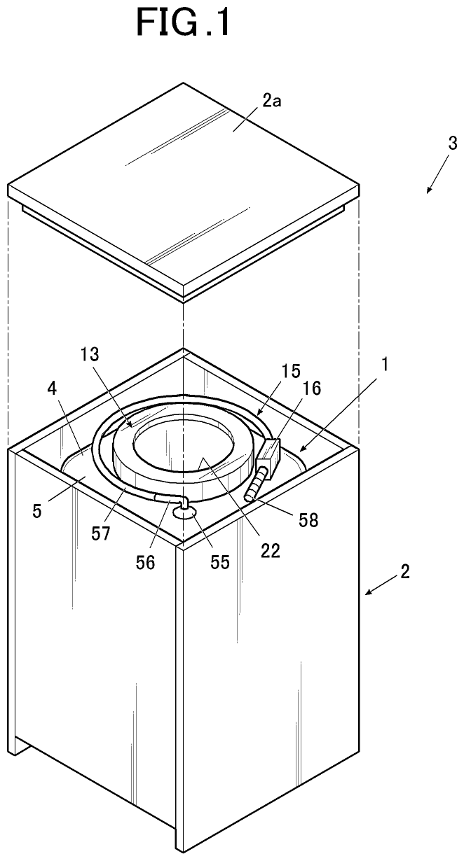

FIG. 1 is a diagram of a hot spring cure box 3 configured with a radon inhalation device 1 stored in a rectangular-parallelepiped box 2 according to a first embodiment of the present invention, and is an external perspective view of the hot spring cure box 3 shown by removing a top panel 2a of the box 2. As shown in FIG. 1, the radon inhalation device 1 according to the present embodiment can be reserved and transported in a state stored in the box 2, and can be used with the top panel 2a of the box 2 being removed. The radon inhalation device 1 according to the present embodiment can be used, reserved, and transported in a state removed from the box 2. The box 2 is mainly made of wood but is not limited thereto. Plastics, metals or the like may be appropriately used.

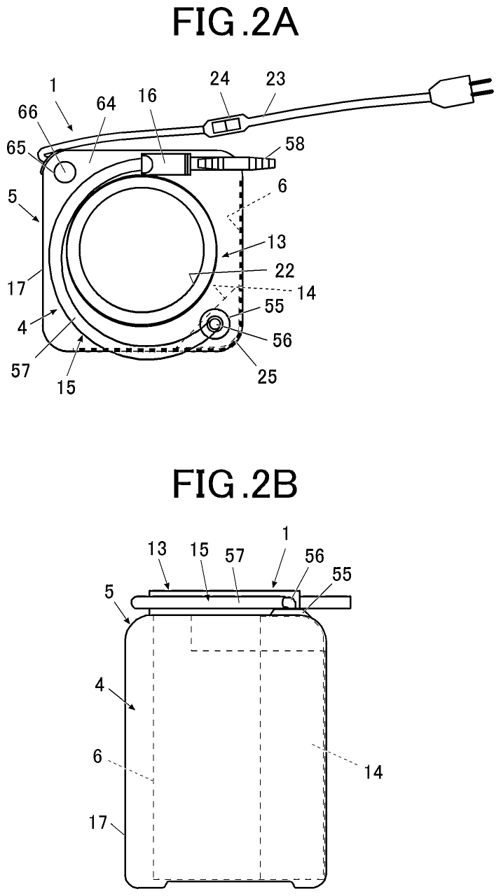

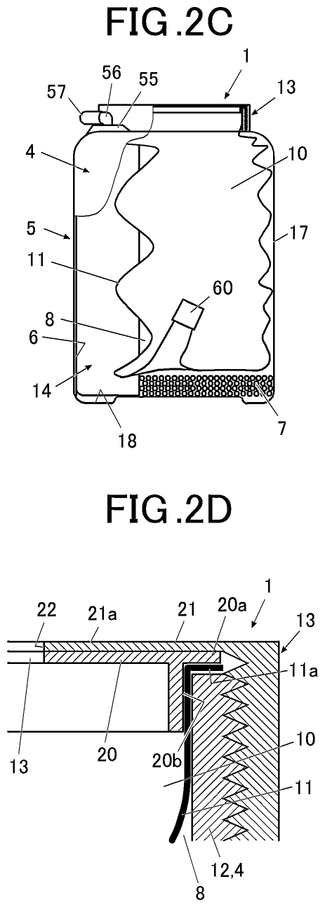

FIGS. 2A to 2D show the radon inhalation device 1 according to the first embodiment of the present invention. FIG. 2A is a plan view of the radon inhalation device 1. FIG. 2B is a frontal view of the radon inhalation device 1. FIG. 2C is a right lateral view of a container main body 4 of the radon inhalation device 1 partially cut out. FIG. 2D is an enlarged view of a part of the radon inhalation device 1 shown in FIG. 2C.

As shown in FIGS. 2A and 2B, the radon inhalation device 1 includes a radon generation container 5 and a heater 6 that heats the inside of the radon generation container 5. The radon generation container 5 includes: the container main body 4 storing radioactive material containing ores 7 (e.g., dried radium ores) and the heater 6; a bag 11 that divides the inside of the container main body 4 into the radon reservoir 8 and the external air opening chamber 10; a bag fixer 13 that is removably attached to an opening 12 of the container main body 4; a radon introducer 14 that communicates with the radon reservoir 8 and the outside of the container main body 4; a gas channel 15 that is attached to the radon extractor 14; and a valve 16 that opens and closes the gas channel 15.

The container main body 4 includes a bottomed cylindrical part 17 in a rectangular parallelepiped shape and the opening 12 formed integrally with the bottomed cylindrical part 17. A bottom circumferential groove 18 is formed along the circumferential rim of the bottom of the bottomed cylindrical part 17. The bag fixer 13 that fixes an opening end 11a of the bag 11 is removably attached to the opening 12. The bag fixer 13 includes an inner fixture 20 and an outer fixture 21. The inner fixture 20 fixes the opening end 11a of the bag 11 to the opening 12, as the outer circumference 20b of the inner fixture 20 is removably fitted to the inner surface of the opening 12 of the container main body 4 with a collar 20a being hooked on the end face of the opening 12 of the container main body 4 and the opening end 11a side of the bag 11 is sandwiched between the end face and the inner surface of the opening 12. The outer fixture 21 fixes the inner fixture 20 and the opening end 11a side of the bag 11 to the container main body 4 as a female thread formed on the inner circumference of the outer fixture 21 is screwed to a male thread formed on the outer circumference of the opening 12 and the collar 20a of the inner fixture 20 and the opening end 11a side of the bag 11 are tacked to the end face of the opening 12. The inner fixture 20 and the outer fixture 21 of the bag fixer 13 have an operation hole 22 with the same diameter opening concentrically with the opening 12 of the container main body 4.

As shown in FIG. 2A, a heater (panel heater) 6 in a sheet form, which stretches over the adjacent walls of the container main body 4, is stored inside the container main body 4. The heater 6, which is flexibly deformable, is inserted inside the container main body 4 from the opening 12 of the container main body 4 and placed such that the center of the heater 6 is positioned at a corner where the radon extractor 14 is placed. The heater 6 is connected to an external power source via an energizing cable 23 and generates heat by being energized when a power switch 24 is turned on to heat the inside of the container main body 4. The heater 6 is not limited to the panel heater that is stored inside the container main body 4 as in the present embodiment. The heater 6 may be any heater that can heat the inside of the radon generation container 5, and may be a heater placed outside of the radon generation container 5.

As shown in FIG. 2C, the bag 11 is formed of a thin-film synthetic resin or thin-film rubber so that it can be deformed easily, and is in a bottomed cylindrical shape with an opening end 11a. The bag 11 is fixed to the opening 12 of the container main body 4 with the opening end 11a being open to divide the inside of the container main body 4 into the radon reservoir 8 and the external air opening chamber 10. That is, in FIGS. 2C and 2D, the bag 11, fixed to the opening 12 of the container main body 4, forms the radon reservoir 8 between its outer circumference of the bag 11 and the inner side of the container main body 4 and forms the external air opening chamber 10 on its inner surface side. The bag 11 is preferably transparent so that other contents in the container main body 4 (e.g., the heater 6, the radon extractor 14) can be observed through the operation hole 22 of the bag fixer 13.

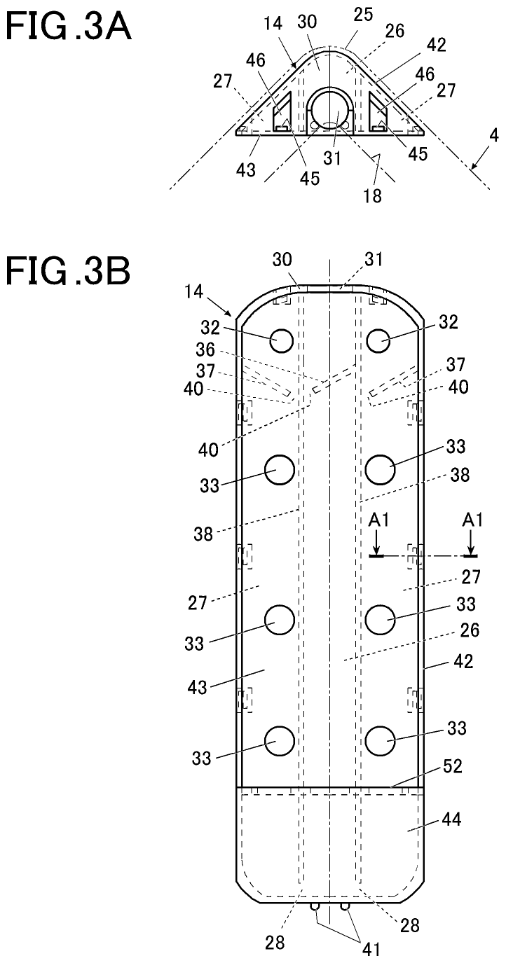

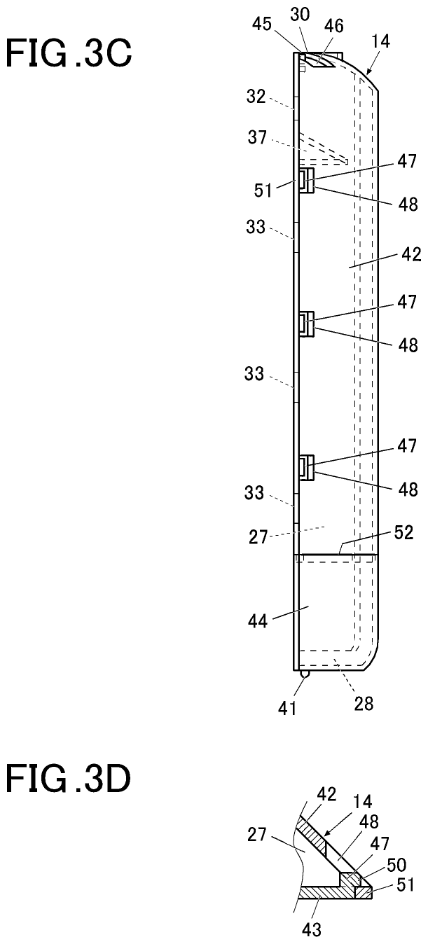

FIGS. 3A to 3D show the radon extractor 14 constituting the radon inhalation device 1 according to the first embodiment of the present invention. FIG. 3A is a plan view of the radon extractor 14. FIG. 3B is a frontal view of the radon extractor 14. FIG. 3C is a right lateral view of the radon extractor 14. FIG. 3D is an enlarged cross-sectional view of the radon extractor 14 of FIG. 3B cut out along a line A1-A1.

As shown in FIGS. 2A to 2D and 3A to 3D, the radon extractor 14 is placed inside the container main body 4 at the corner 25 where the heater 6 is installed, and is substantially in an isosceles right-angled triangle in a plan view. The radon extractor 14 includes parallel channels 26, 27. The parallel channels 26, 27 of the radon extractor 14 is formed of a center channel 26 and a pair of the side channels 27, 27 positioned on both sides of the center channel 26, and the lower part of the center 26 and the lower part of the pair of the side channels 27, 27 communicate with each other via the connection port 28. The center channel 26 communicates with the outside of the container main body 4 via the gas channel 15 attached to a gas channel attachment hole 31 formed at a top 30 of the radon extractor 14. The pair of the side channels 27, 27 respectively communicate with the radon reservoir 8 via vents 32, 33.

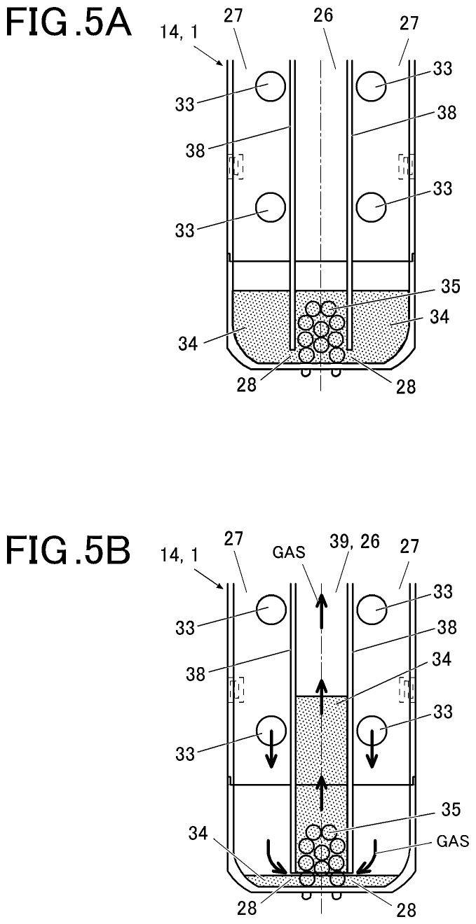

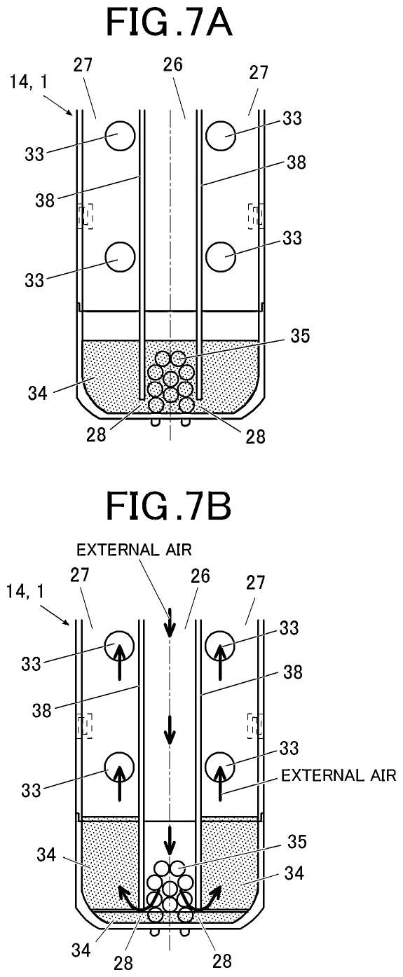

As shown in FIG. 3A to 3D, in the radon extractor 14, the liquid level of a liquid 34 is above the connection port 28 in a normal state (when there is no difference between the inside pressure of the radon reservoir 8 and the outer pressure of the container main body 4) and the liquid level of the center channel 26 and the liquid level of the pair of the side channels 27, 27 are the same, as the liquid 34 is stored in the lower part of the parallel channels (the center channel 26 and the pair of the side channels 27, 27). The connection port 28 is thus blocked by the liquid 34 (see FIGS. 5A and 7A). Filtering gravel 35 is stored at the lower part of the center channel 26 in the radon extractor 14 (see FIGS. 5A, 5B, 7A, and 7B). The liquid 34 stored inside the radon extractor 14 may be water, but is preferably a liquid with a color such as green tea, because the remaining amount can be easily checked and has a sterilizing effect.

As shown in FIGS. 3B and 3C, in the radon extractor 14, splash prevention walls 36, 37 that prevent splashing are formed obliquely downward at the upper part of the center channel 26 and the upper part of the pair of the side channels 27, 27, and a gap 40 is formed between each end of the splash prevention walls 36, 37 and a channel wall 38.

As shown in FIGS. 3B and 3C, the radon extractor 14 has a pair of support protrusions 41, 41 formed on the lower surface, and is supported in a state positioned at the corner 25 inside the container main body 4 with the pair of the support protrusions 41, 41 being engaged with the bottom circumferential groove 18 of the container main body 4.

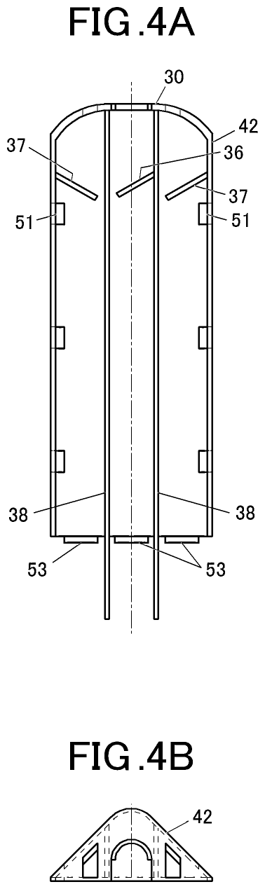

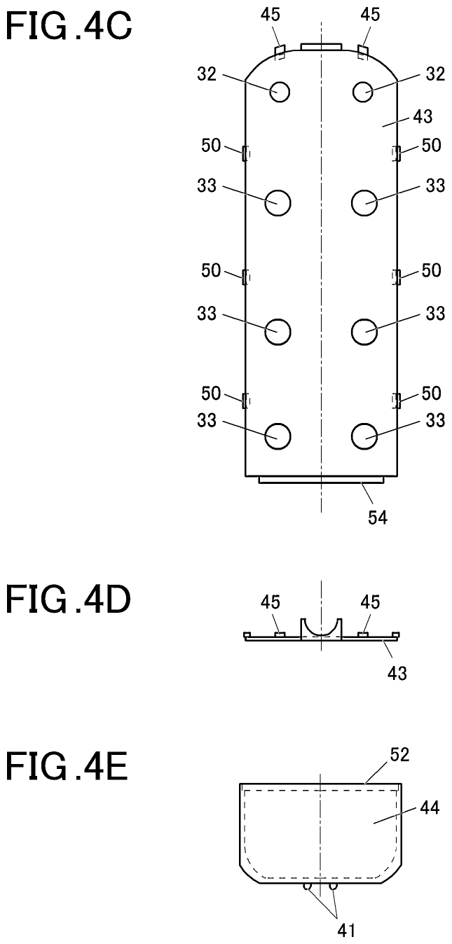

FIGS. 4A to 4E are exploded views of the radon extractor 14. As shown in FIGS. 4A to 4E, the radon extractor 14 includes a shell 42, a substantially plate-shaped frontal wall 43 that blocks the frontal face of the shell 42, and a bottomed cylindrical bottom lid member 44 that fits to the lower end of the shell 42 and the frontal wall 43. In the radon extractor 14, a pair of tongue-shaped first engaging protrusions 45 at the upper part of the frontal wall 43 is fitted to a pair of first catching holes 46 formed at the upper part of the shell 42, and a plurality of second engaging protrusions 47 formed along the both end peripheries of the frontal wall 43 is fitted to a plurality of second catching holes 48 formed along the lateral rim of the shell 42 (in the same number as the second engaging protrusions 47). The hooks 50 of the second engaging protrusions 47 are thereby hooked on opening edges 51 of the second catching holes 48 and the shell 42 and the frontal wall 43 are attach/detachably (separably) integrated by the elastic force of the second engaging protrusions 47. In this state, in the radon extractor 14, the end face of the channel wall 38 dividing the center channel 26 and the side channels 27 of the shell 42 is in contact with the inner surface of the frontal wall 43 and the center channel 26 and the side channels 27 are separated by the channel wall 38 and the frontal wall 43. The radon extractor 14 is assembled with the shell 42 and the frontal wall 43. In the state where the radon extractor 14 before the bottom lid member 44 is assembled to the shell 42 and the frontal wall 43, the pair of the channel walls 38, 38 protrudes (is exposed) downward from the shell 42 and the frontal wall 43.

As shown in FIGS. 4A to 4E, the radon extractor 14 is sealed with the bottom lid member 44 on the lower side of the shell 42 and the frontal wall 43 as the opening end 52 side of the bottom lid member 44 is fitted to thin-walled parts 53, 54 on the lower side of the shell 42 and the frontal wall 43. In the radon extractor 14, a part of the end face of the pair of the channel walls 38, 38 which protrudes downward from the shell 42 and the frontal wall 43 is in contact with the inside of the bottom lid member 44, which makes it possible to suppress gas flow between the center channel 26 and the side channel 27 except through the connection port 28.

As shown in FIGS. 2A to 2C, the gas channel 15 includes: an L-shaped elbow tube 56 attached with a bushing 55 bridged over both the gas channel attachment hole 31 formed at a top part 30 of the radon extractor 14 and a through hole (not shown) formed at a position aligning with the gas channel attachment hole 31 in an upper wall part 64 of the container main body 4; an elastically deformable tube 57 connected to the elbow tube 56; and a suction member 58 removably fitted to the end of the tube 57. The simple valve 16 for opening/closing the channel is attached to the gas channel 15 on the end side of the tube 57, and the circulation of the gas in the flow path (the tube 57) is stopped by closure of the valve 16 and the circulation of the gas in the flow path (the tube 57) is enabled by opening of the valve 16. The bushing 55, which is attached to the corner part of the upper wall part 64 of the container main body 4, causes the periphery of the gas channel attachment hole 31 of the radon extractor 14 to adhere to the periphery of the through hole on the container main body 4 and blocks the gap between the elbow tube 56 and the container main body 4 to prevent the gas in the container main body 4 from leaking to the outside of the container main body 4.

FIGS. 5A and 5B show a first use state of the radon inhalation device 1 according to the present embodiment. FIG. 5A shows the state of use of the radon inhalation device 1 before radon inhalation. FIG. 5B shows a state of use of the radon inhalation device 1 during radon inhalation.

As shown in FIGS. 2A to 2D and 5A, in the radon inhalation device 1, the inside of the container main body 4 is heated by the heater 6, which causes part of the liquid 34 in the radon extractor 14 to become water vapor, raising the humidity in the radon reservoir 8. The radon released from the radioactive material containing ores 7 thereby accumulates in the radon reservoir 8. Then, in the radon inhalation device 1, as shown in FIGS. 2A to 2D and 5B, as a user inhales the gas in an internal space 39 of the center channel 26 in the radon extractor 14 via the gas channel 15: the liquid level of the center channel 26 rises; the liquid level of the pair of the side channels 27, 27 falls to a level a little lower than the upper edge of the connection port 28; when a small gap appears between the liquid surface of the pair of the side channels 27, 27 and the upper edge of the connection port 28, a pressure rapidly falls at a part between the liquid 34 lifted in the center channel 26 and the liquid 34 remaining on the bottom side of the radon extractor 14; the surrounding gas rapidly flows into the part where the pressure rapidly falls; and the gas rapidly flows into the liquid 34 in the center channel 26 like being blown. As a result, in the radon inhalation device 1, the gas containing the radon passes through the liquid 34 in the center channel 26 of the radon extractor 14 as bubbles, and is discharged at the top of the liquid 34 of the center channel 26 like popping. At this time, the liquid splashing on the liquid surface hits the liquid splash prevention wall 36 of the center channel 26 and falls, and therefore it does not flow into the gas channel 15. In the radon inhalation device 1, the gas containing radon passes through the gap between the liquid splash prevention wall 36 and the channel wall 38, and a user inhales the gas containing radon via the gas channel 15. At this time, unwanted materials such as minute particle in the gas passing through the liquid 34 in the center channel 26 are trapped in the liquid 34.

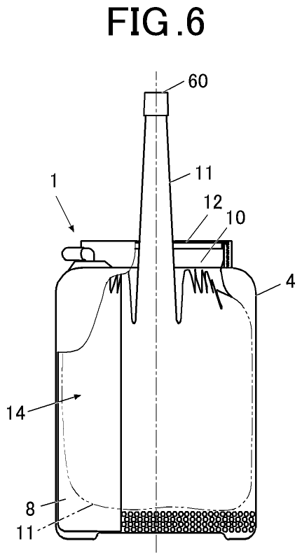

FIG. 6 shows a second use state of the radon inhalation device 1 with the container main body 4 partially cut out. As shown in FIG. 6, in the radon inhalation device 1, in a case where the gas inside the radon reservoir 8 decreases and the volume of the radon reservoir 8 divided by the bag 11 decreases (if the bag 11 is deformed as shown in two dotted line), as the user inserts their hand through the operation hole 22 of the bag fixer 13 fixed to the opening 12 of the container main body 4 (see FIGS. 2A to 2D) and lifts the bag 11 holding its operation part 60, the volume of the radon reservoir 8 rapidly increases and the pressure inside the radon reservoir 8 rapidly decreases. As a result, in the radon inhalation device 1, the liquid 34 in the side channels 27 of the radon extractor 14 rapidly rises and the liquid 34 in the center channel 26 of the radon extractor 14 rapidly falls (see FIG. 7B).

FIGS. 7A and 7B show a third use state of the radon inhalation device 1 according to the present embodiment. FIG. 7A shows the state of use of the radon inhalation device 1 before introduction of external air. FIG. 7B shows a state of use of the radon inhalation device 1 during introduction of external air.

As shown in FIGS. 7A and 7B, in the radon inhalation device 1, as the lower level of the liquid 34 inside the center channel 26 of the radon extractor 14 moves to the upper edge of the connection port 28 and a small gap appears between the lower level of the liquid 34 and the liquid 34 remaining on the bottom side of the radon extractor 14, the pressure rapidly decreases at the gap and the gas in the center channel 26 flows into the liquid 34 in the side channels 27 through the connection port 28 like being blown. The gas flowing into the side channels 27 like being blown rises through the liquid 34 in the side channel 27 as bubbles, and is discharged at the top of the liquid 34 of the side channels 27 like popping. At this time, the liquid 34 splashing on the liquid surface hits the liquid splash prevention wall 37 of the side channel 27, being prevented from flowing into the radon reservoir 8 (see FIGS. 3B and 3C). The radon inhalation device 1 is not limited to the embodiment shown in FIGS. 3B and 7B, and the vents 33 of the side channels 27 may be omitted. The radon inhalation device 1 is not limited to the embodiment shown in FIGS. 3B and 7B, and the liquid splash prevention wall 37 may be equipped at a part lower than the vents 33 which are positioned at the lowest part of the side channels 27.

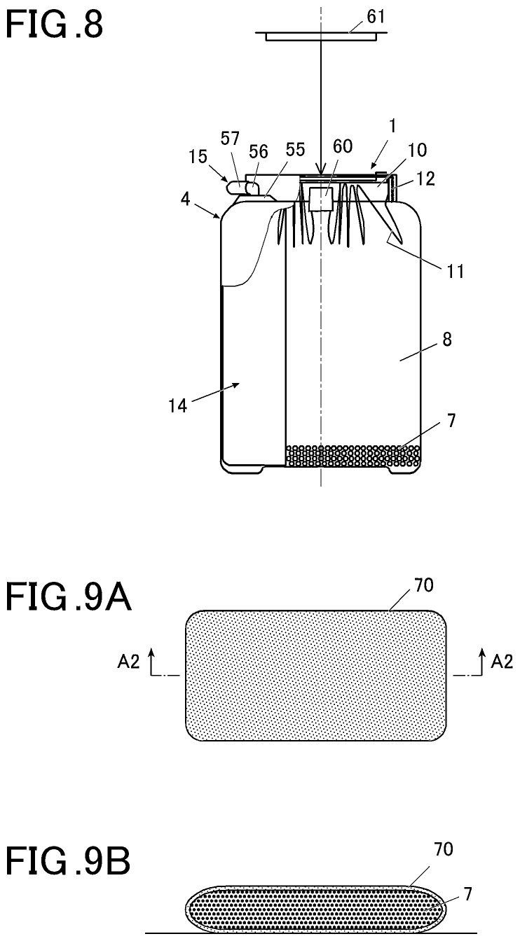

FIG. 8 shows a fourth use state of the radon inhalation device 1 according to the present embodiment with the container main body 4 partially cut out.

As shown in FIG. 8, in a state where the radon inhalation device 1 has finished introducing external air into the radon reservoir 8, the bag 11 is pushed to the vicinity of the opening 12 of the container main body 4, being squeezed by the introduced external air and most of the inside of the container main body 4 becomes the radon reservoir 8. In the radon inhalation device 1, when the radon is reserved in the radon reservoir 8, the valve 16 of the gas channel 15 is closed and the circulation of the gas inside the gas channel 15 is blocked (see FIG. 2A). A lid member 61 is preferably engaged to the operation hole 22 of the inner fixture 20 and the outer fixture 21 of the bag fixer 13 attached to the opening 12 of the container main body 4 to prevent foreign materials from falling inside the container main body 4 (see FIG. 2D).

In the state shown in FIG. 8 of the radon inhalation device 1, the inside of the container main body 4 is heated by the heater 6, the vapor generated from the liquid 34 inside the radon extractor 14 flows into the radon reservoir 8. And when the temperature and humidity inside the radon reservoir 8 get appropriate, the radon is effectively released from the radioactive material containing ores 7 to the radon reservoir 8.

In the radon inhalation device 1 according to the present invention described above, as the inside of the radon generation container 5 can be heated with the heater 6 to reach the temperature appropriate for radon production, users can inhale radon at any time at a stable concentration regardless of the surrounding atmospheric temperature to gain effects similar to those of radon hot spring.

As shown in FIGS. 5B and 7B, in the radon inhalation device 1 according to the present embodiment, as the filtering gravel 35 is stored at the lower part of the center channel 26 of the radon extractor 14, the bubbles passing through the liquid in the center channel 26 can be prevented from getting bigger by the filtering gravels 35, which further suppress liquid splash. In the radon inhalation device 1 according to the present embodiment, what is stored at the lower part of the center channel 26 is not limited to the filtering gravel 35, and may be anything that is expected to have the same effects as the filtering gravel 35, such as a stainless steel brush, for example.

In the radon inhalation device 1 according to the present embodiment, as shown in FIG. 5B, the liquid splashes when bubbles of the gas passing through the liquid in the center channel 26 are collapsed on the liquid surface, but the liquid splashing on the liquid surface hits the liquid splash prevention wall 36, being prevented from flowing into the gas channel 15.

In the radon inhalation device 1 according to the present embodiment, the pair of the side channels 27, 27 is formed in the radon extractor 14, but the present invention is not limited to this example, and one of the side channels 27, 27 may be omitted.

In the present embodiment, as unwanted materials such as minute particles are trapped by the liquid 34, there is not much necessity for a filter, but in order to improve the effectiveness of trapping unwanted materials, a filter may be provided in the radon extractor 14 or/and the gas channel 15 as necessary.

### Variation 1 of First Embodiment

As shown in FIG. 2A, in the radon inhalation device 1, an external air intake 65 through which the inside of the radon reservoir 8 and the outside of the container main body 4 communicate is formed on the upper wall part 64 pf the container main body 4 surrounding the opening 12 of the container main body 4. The external air intake 65 is positioned at the corner part of the upper wall part 64 of the container main body 4 facing the bushing 55, and is blocked and opened with a stopper 66 (e.g., rubber stopper, plastic stopper) being fitted thereto and being removed therefrom.

In the radon inhalation device 1 according to the present variation, as shown in FIGS. 6 and 7B, in a case where external air is to be introduced into the radon reservoir 8 of the container main body 4, external air can be introduced into the radon reservoir 8 more easily and in a shorter time the external air intake 65 when the stopper 66 is removed from the external air intake 65 and the external air intake 65 is opened in comparison to when external air is introduced into the radon reservoir 8 through the gas channel 15 and the radon extractor 14 only, because external air can be introduced into the radon reservoir 8 through the gas channel 15 and the radon extractor plus the external air intake 65.

The radon inhalation device 1 according to the present variation, the amount of external air introduced via the radon extractor 14 can be suppressed, and liquid splashing on the liquid surface of the side channels 27 can thereby be suppressed (see FIG. 7B).

In the radon inhalation device 1 according to the present variation, after external air is introduced to the radon reservoir 8, the external air intake 65 is blocked by the stopper 66.

### Variation 2 of First Embodiment

In the radon inhalation device 1 according to the first embodiment, the radioactive material containing ores 7 are directly spread inside the radon generation container 5, but the radioactive material containing ores 7 are preferably powdered so that radon is effectively released for the surface area of the radioactive material containing ores 7 to be enlarged. In a case where the radioactive material containing ores 7 are powdered, radon can be effectively released to the radon reservoir 8, but the powders easily enter the radon extractor 14. Therefore, as shown in FIGS. 9A and 9B, the powdered radioactive containing ores 7 are preferably put into a bag 70 made of non-woven fabric or the like to be stored in the radon reservoir 8.

### Variation 3 of First Embodiment

In the first embodiment, the gas channel 15 attached to the radon extractor 14 is equipped with the valve 16 to open/close it, and a filter is provided as necessary in the radon extractor 14 and/or the gas channel 15. However, when the gas containing radon is inhaled from a radon extractor 62 via the gas channel 15, the valve 16 and a filter may easily act as a ventilation resistance. Thus, for those with weak inhalation, there will be a concern that the gas containing radon cannot be inhaled sufficiently.

Accordingly, the radon extractor 14 and the gas channel 15 connected thereto according to the first embodiment are not necessarily equipped with a valve or a filter so that a ventilation resistance is reduced as much as possible. Even if a valve and a filter are omitted and the gas channel 15 is left open, the radon extractor 14 stores the liquid 34 inside and this stored liquid 34 seals the part between the flow path to the radon reservoir 8 and the flow path to the gas channel 15. Thus, there will not be a problem that the gas containing radon leaks through the gas channel 15.

### Variation 4 of First Embodiment

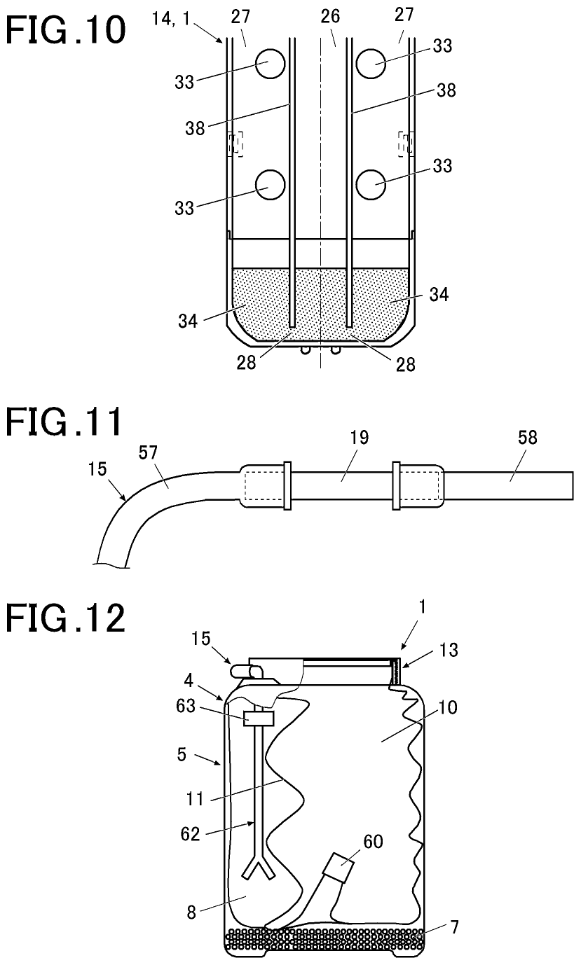

Further, the filtering gravel 35 stored in the radon extractor 14 shown in FIGS. 5A, 5B, 7A, and 7B may be omitted as shown in FIG. 10 to reduce a ventilation resistance of the gas containing radon. In a case where the filtering gravel 35 is omitted, bubbles passing through the liquid may get bigger and splash more easily, but it is easier to inhale the gas containing radon with water vapor and take radon deep inside the human body.

### Variation 5 of First Embodiment

As shown in FIG. 11, the gas channel 15 may be equipped with a joint tube 19 instead of the valve 16, and through the joint tube 19 the suction member 58 may be detachably installed. Assuming that the suction member 58 is frequently replaced, the suction member 58 should be easily replaced using the joint tube 19 provided at the end of the gas channel 15 and connected to the suction member 58 in an easily attachable/detachable manner.

### Second Embodiment

FIG. 12 shows the radon inhalation device 1 according to a second embodiment of the present invention with the container main body 4 partially cut out. In the radon inhalation device 1 according to the present embodiment shown in FIG. 9, the components common to the radon inhalation device 1 according to the first embodiment shown in FIG. 2C are omitted as appropriate. The common components have the same reference signs to those of the radon inhalation device 1 according to the first embodiment shown in FIG. 2C, and the description overlapping with that of the radon inhalation device 1 in the first embodiment is omitted.

As shown in FIG. 12, in the radon inhalation device 1 according to the present embodiment, the radon extractor 62 is different from the radon extractor 14 in the radon inhalation device 1 according to the first embodiment.

The radon extractor 62 in the radon inhalation device 1 according to the present embodiment is a cylindrical body with its tip branched into two, and is equipped with a filter 63 at its upper part connected to the gas channel 15. In this radon inhalation device 1, when the user inhales the gas containing radon inside the radon reservoir 8 via the gas channel 15, unwanted materials are filtered out by the filter 63.

The radon inhalation device described above according to the present embodiment has similar effects as those of the radon inhalation device according to First Embodiment.

### Third Embodiment

The radon inhalation device 1 according to the first and second embodiments has the heater 6 that heats the inside of the radon generation container 5 placed inside the container main body 4 for example, but is not limited thereto. The inside of the radon generation container 5 may be heated by a heater placed outside the radon generation container 5 (e.g., in the box 2, the lower side of the exterior of the container main body 4 or the lateral side of the exterior of the container main body 4).

### Fourth Embodiment

lighting means (e.g., LED lighting device) for visually checking the remaining amount of the liquid 34 in the radon extractor 14, the state of the filtering gravel 35, the state of the radioactive material containing ores 7, and the like may be provided inside the container main body 4.

The lighting means can be attached at a position facing the radon extractor 14 at substantially the center of the inside of the container main body 4.

### Fifth Embodiment

In the radon inhalation device 1 according to the first embodiment, the center channel 26 is connected to the gas channel 15 so that the side channels 27 communicate with the radon reservoir 8, but the configuration is not limited to this. The side channels 27 may be connected to the gas channel 15 so that the center channel 26 communicates with the radon reservoir 8. In this embodiment, the vents 32, 33 are formed in the center channel 26 and not in the side channels 27. Furthermore, in this embodiment, the filtering gravel 35 is preferably positioned at the lower part of the side channels 27.

### Sixth Embodiment

In the configurations described above, the radon inhalation device 1 with a small capacity (e.g., a device with a capacity of the radon generation container 5 of 5 L) is assumed as an example. However, in the radon inhalation device 1 with a large capacity, in the radon inhalation device 1 with a capacity of the radon generation device 5 of 50 L, for example, the bag 11 gets larger. When the capacity of the radon reservoir 8 divided by the bag 11 is reduced, it is hard for the user to lift the bag 11 by inserting the hand into the container main body 4. In a case where the radon inhalation device 1 is installed in a commercial facility, a hotel, or the like, it is undesirable that the inside of the radon inhalation device 1 is not operated by a number of unspecified users.

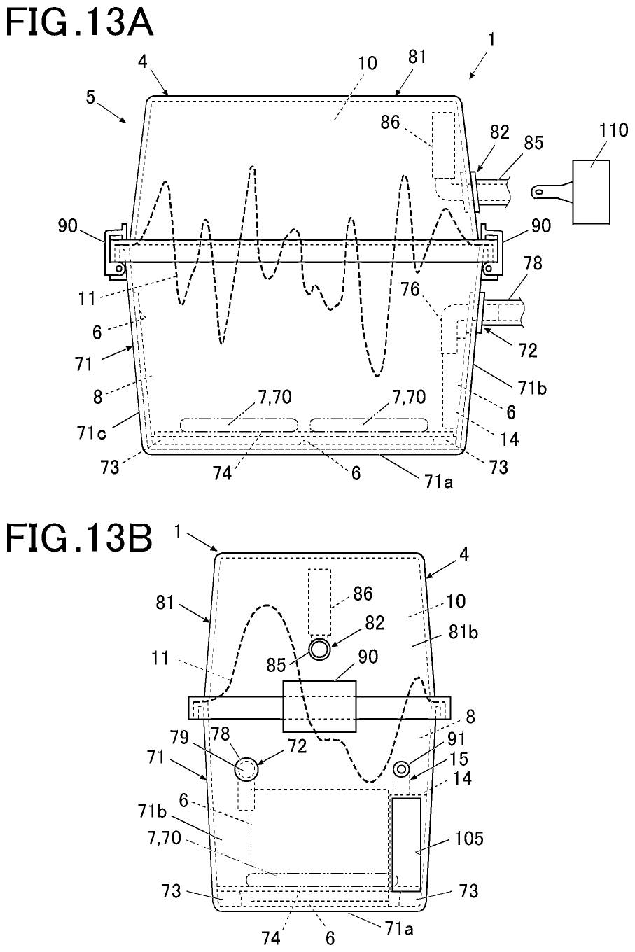

As shown in FIGS. 13A and 13B, it is preferable that the gas in the external air opening chamber 10 is suctioned by an air suction machine 110 to depressurize the external air opening chamber 10 to lift the bag 11 so that the capacity of the radon reservoir 8 may be increased without operating the bag 11 by a user inserting the hand therein.

Specifically, the radon generation container 5 includes: the container main body 4 formed with a lower case member 71 storing therein the radioactive material containing ores 7 (e.g., dried radium ores or the like powdered and stored in the bag 70 made of a non-woven fabric), the heater 6, and the like, and an upper case member 81 as a cover member removably attached to the lower case member 71; the bag 11 dividing the space inside the container main body 4 (the space surrounded by the lower case member 71 and the upper case member 81) into the radon reservoir 8 and the external air opening chamber 10; the radon extractor 14 that communicates with the radon reservoir 8 and the outside of the container main body 4; the gas channel 15 attached to the radon extractor 14; an air suction channel 82 to which the air suction machine 110 suctioning the air in the external air opening chamber 10 is attachable; and an external air introducing channel 72 that can introduce external air to the radon reservoir 8.

The lower case member 71 is formed in a substantially rectangular parallelepiped shape with an open top, and the upper case member 81 is formed in a substantially rectangular parallelepiped shape with an open bottom. The lower case member 71 and the upper case member 81 are assembled with their respective open ends facing each other to form the container main body 4. The lower case member 71 and the upper case member 81 are preferably made of a transparent or translucent material so that the inside is visible. A locking mechanism 90 of a buckle or the like is preferably provided to fix the state where the lower case member 71 is assembled to the upper case member 81.

The bag 11 is preferably made of a synthetic resin or thin-film rubber, similarly to the former embodiments, so that it can be easily deformed, and is in a bottomed cylindrical shape. As the bag 11 is fixed with its opening end being open between the upper opening of the lower case member 71 and the lower opening of the upper case member 81, the space surrounded by the lower case member 71 and the upper case member 81 is divided into the radon reservoir 8 and the external air opening chamber 10.

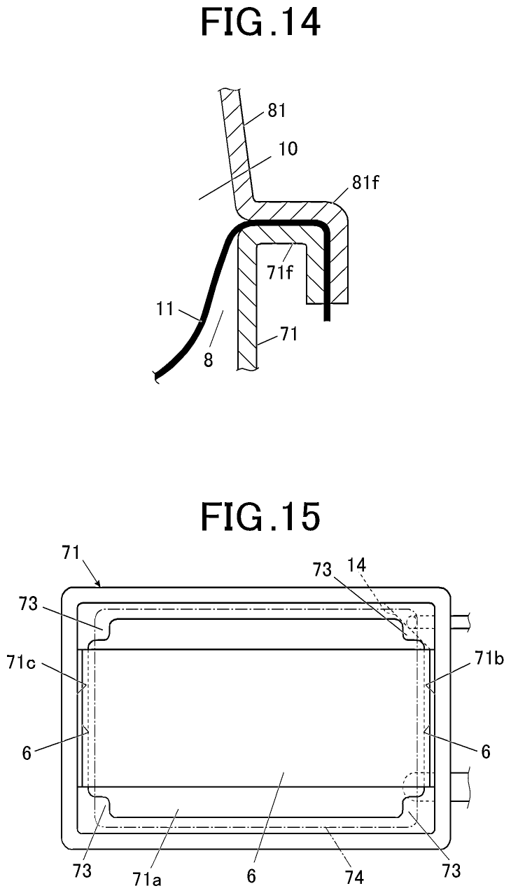

That is, as shown in FIG. 14, the periphery of the opening end of the bag 11 is tucked between a male flange 71f formed protruding outward at the periphery of the upper opening of the lower case member 71 and a female flange 81f formed protruding outward at the periphery of the lower end of the upper case member 81 to be fitted to the male flange 71f, and in order to maintain this state, the lower case member 71 and the upper case member 81 are locked with the lock mechanism 90. Further, the radon reservoir 8 is formed between the inner surface of the lower case member 71 and the bag 11 (at a part lower than the bag 11 in the space surrounded by the lower case member 71 and the upper case member 81), and the external air opening chamber 10 is formed between the inside of the upper case member 81 and the bag 11 (at a part upper than the bag 11 in the space surrounded by the lower case member 71 and the upper case member 81).

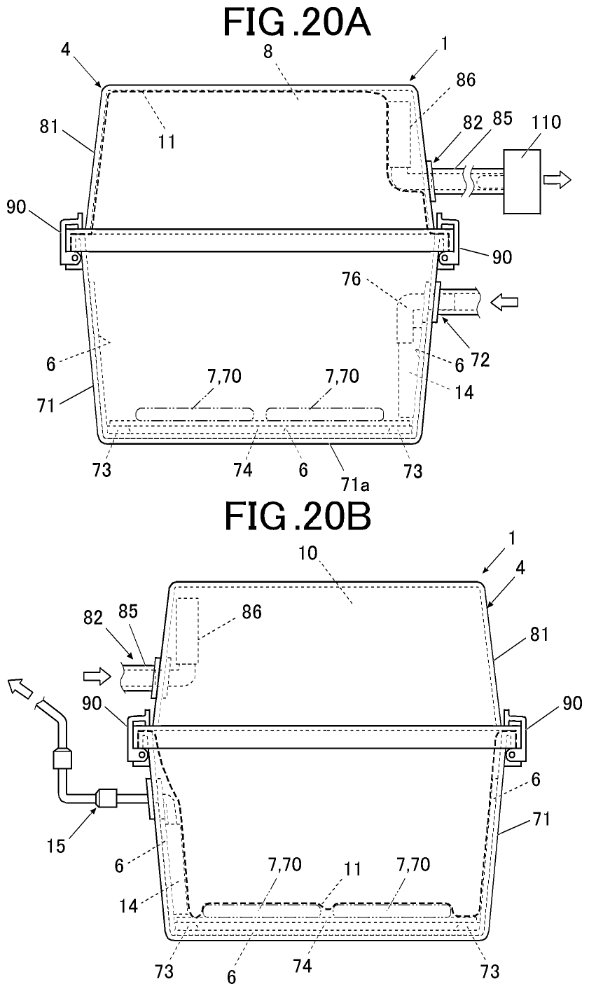

The bag 11 can be shifted vertically by the part sandwiched between the male flange 71f and the female flange 81f, as the bag 11 is shifted from the state in which its bottom is brought to the inner bottom wall of the lower case member 71 or the vicinity thereof to the state in which its bottom is brought to the inner top wall of the upper case member 81 or the vicinity thereof. That is, the bag 11 can be moved inside the case to cause the inside of the case to be almost entirely the external air opening chamber 10 (the state shown in FIG. 20B described later) and cause the inside of the case to be almost entirely the radon reservoir 8 (the state shown in FIG. 20A described later).

As shown in FIG. 15, the lower case member 71 includes a protrusion 73 protruding upward at the four corners of a bottom wall 71a, and is provided with a heater (panel heater) 6 in a sheet form of a predetermined width on the inner surface of the pair of lateral walls 71b, 71c facing each other and on the upper surface of the bottom wall 71a, the heater avoiding the protrusion 73. As described above, with the heater 6 placed from the inner surface of one of the pair of lateral walls 71b, 71c facing each other to the inner surface of the other one of the pair of lateral walls 71b, 71c facing each other via the upper surface of the bottom wall 71a (as the heater is installed on the three sides of the lower case member 71 of: the inner surfaces of the lateral walls 71b, 71c; and the upper surface of the bottom wall 71a), even when the container main body 4 with a large capacity is used, the entire inside of the container can be evenly and effectively heated and temperature unevenness in the container is not easily caused by the surrounding atmospheric temperature. As a result, radon at a stable concentration can be provided at any time regardless of the surrounding atmospheric temperature.

An aluminum plate 74 (shown by a dot line in FIG. 15) is placed above the heater spreading along the bottom wall 71a to bridge the protrusions 73 at the four corners of the bottom wall 71a. The radioactive material containing ores 7 are disposed on the upper surface of the aluminum plate 74. The radioactive material containing ores 7 are powdered and stored in the bag 70, and a necessary number of the bags 70 are placed on the aluminum plate 74.

At the corners inside the lower case member 71, the radon extractor 14 shown in FIGS. 3A to 3D and 4A to 4E is fixed with an appropriate means such as double-faced tape, and the gas channel 15 attached to the top 30 of the radon extractor 14 is stretched out of the container main body 4. In this example, the radon extractor 14 is installed without interfering with the heater 6. The heater 6 is provided over the three sides of the lower case member as described above to have a large surface area and sufficient heating capacity. The inside of the container main body 4 can be sufficiently warmed without the radon extractor 14 being in direct contact, making it possible to warm the liquid inside the radon extractor and increase the humidity.

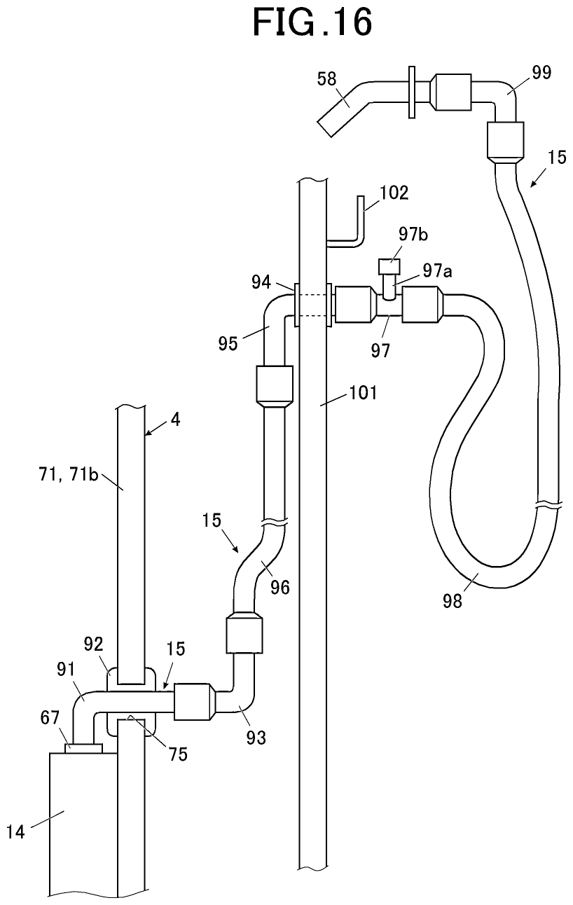

The extractor 14 used here is the same as that shown in the first embodiment (FIGS. 3A to 3D, 4A to 4E) and the description thereof is omitted. However, the gas channel 15, as shown in FIG. 16, has an L-shaped joint member (elbow joint 91) attached with a bushing 67 to the gas channel attachment hole 31 (see FIGS. 3A and 3B) formed at the top 30 of the radon extractor 14, and the elbow joint 91 penetrates through the through hole 75 formed on the lateral wall 71b of the lower case member 71 via the bushing 92 to protrude outward from the lateral wall 71b of the lower case member 71.

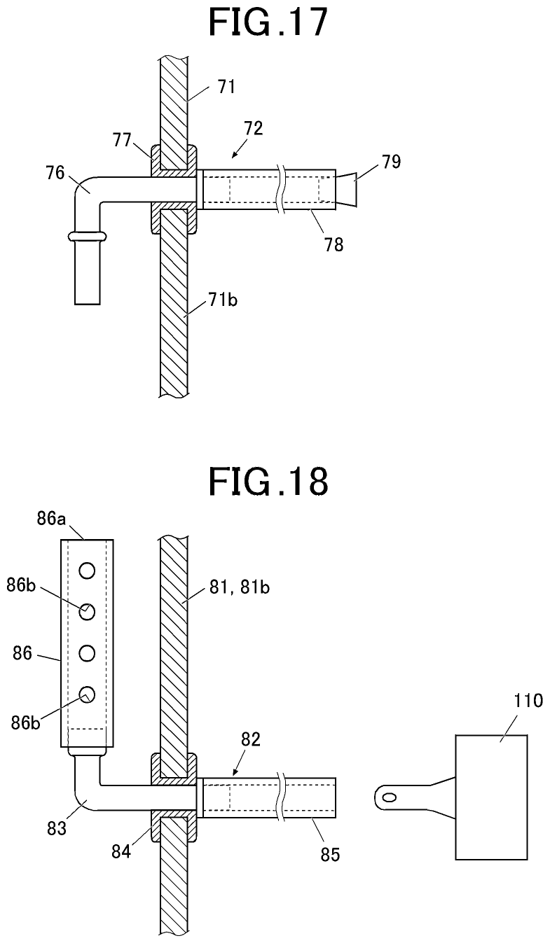

An L-shaped elbow joint 76 as a joint member that forms part of the external air introducing channel 72 is attached to the lateral wall 71b of the lower case member 71 to penetrate therethrough via a bushing 77, as shown in FIG. 17. The external air introducing channel 72 is formed of the elbow joint 76 and an external air introducing pipe 78 connected to the part, of the elbow joint 76, protruding outside of the lower case member 71. The part, of the elbow joint 76, protruding inside of the case is bent downward and is extended downward along the inner surface of the lateral wall 71b of the lower case member 71 so as not to obstruct the movement of the bag 11. The opening end of the external air introducing pipe 78 is detachably sealed with a stopper 79.

On the other hand, at the substantial center of a lateral wall 81b (see FIGS. 13A and 13B) of the upper case member 81 positioned directly above the lateral wall 71b through which the gas channel 15 and the external air introducing channel 72 of the lower case member 71 penetrate, an L-shaped elbow joint 83 as a joint member that forms part of the air suction channel 82 is attached to the upper case member 81 to penetrate therethrough via a bushing 84, as shown in FIG. 18. The air suction channel 82 includes the elbow joint 83, an air suction pipe 85 connected to the part, of the elbow joint 83, protruding outside of the upper case member 81, and a support pipe 86 connected to the part, of the elbow joint 83, protruding inside of the upper case member 81.

In order to prevent air suction from being blocked by attachment of the bag 11 as well as not to obstruct the movement of the bag 11 inside the container main body 4, the part, of the elbow joint 83, protruding inside the upper case member 81 is bent upward substantially at 90 degrees and the support pipe 86 is extended upward along the inner surface of the lateral wall 81b of the upper case member 81. The shape of the support pipe 86 is not limited, but an end opening 86a thereof is directed upward to prevent the bag 11 from attaching to and blocking the end opening even when the air inside the external air opening chamber 10 is suctioned from the outside of the case via the air suction pipe 85. A plurality of through holes 86b are formed at appropriate spots on the peripheral surface of the support 86 so that the air can be suctioned through the through holes 86b, and the air can be suctioned through the through holes 86b on the periphery of the support pipe 86 even when the end opening 86a is blocked by the bag 11.

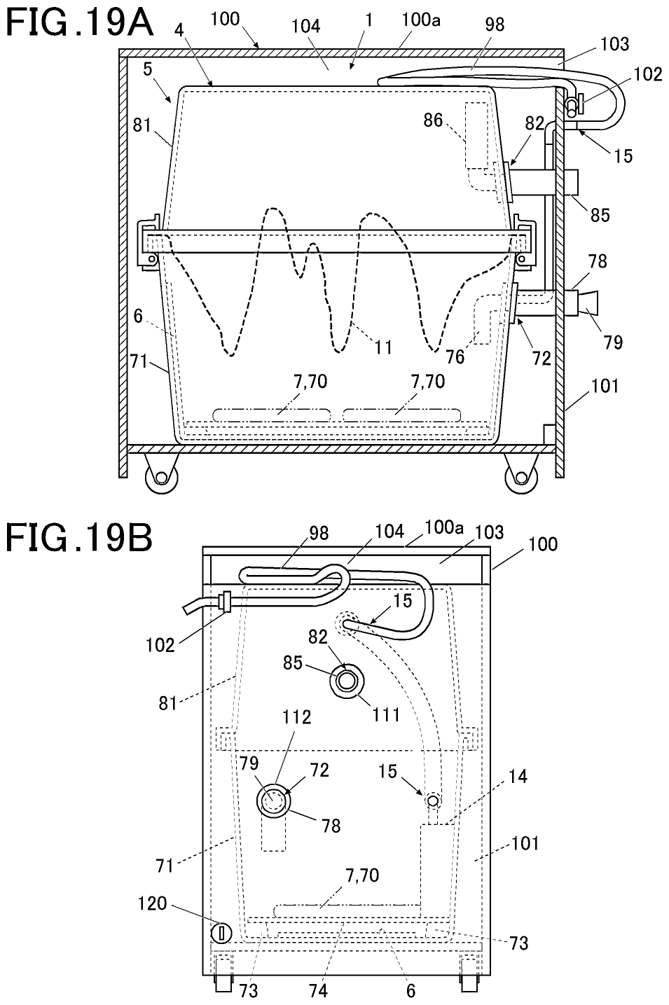

The radon inhalation device 1 described above can be used in a state where it is installed in a cabinet 100 (bedside cabinet) beside a bed in a hotel or the like, and in that case, as shown in FIGS. 19A and 19B, the radon inhalation device 1 is installed inside the cabinet 100 as a door 101 on the front of the cabinet 100 is opened, and the gas channel 15, the external air introducing pipe 78 of the external air introducing channel 72, the air suction pipe 85 of the air suction channel 82 are drawn out in front of the cabinet 100 through the door 101.

The door 101 is a single swinging door which opens/closes with a hinge not shown in the drawings on the left or the right of the front opening of the cabinet 100, and the air suction pipe 85 is drawn out through a through hole 111 formed substantially at the center in the upper part of the door 101 facing the air suction pipe 85. The external air introducing pipe 78 is drawn out through a through hole 112 formed at the part of the door 101 facing the external air introducing pipe 78.

When the door 101 is opened, the air suction pipe 85 and the external air introducing pipe 78 are off the through holes 111, 112 in the door 101, and when the door 101 is closed, the air suction pipe 85 and the external air introducing pipe 78 are inserted through the through holes 111, 112 in the door 101 to protrude forward from the door 101.

When external air is not to be introduced into the radon reservoir 8, the external air introducing pipe 78 is sealed with the stopper 79 being inserted to the end opening. On the other hand, the end opening of the air suction pipe 85 need not be sealed by a stopper, but it may be sealed with a stopper or a cap to prevent dust ingress or the like.

As shown in FIG. 16, the gas channel 15 includes: in addition to the elbow joint 91 fixed on the side wall 71b of the lower case member 71 of the radon generation container 5; an L-shaped first elbow joint 93 connected to a part, of the elbow joint 91, protruding from the side wall 71b and bent at approximately 90 degrees upward; an L-shaped second elbow joint 95 fixed at an upper central part of the door 101 via a bushing 94 and bent at approximately 90 degrees downward on the inside of the door 101; a joint hose 96 connecting the upper end opening of the first elbow joint 93 and the lower end opening of the second elbow joint 95; a T-shaped hose joint 97 connected to a part, of the second elbow joint 95, protruding outside of the door 101; a long flexible tube 98 connected via the T-shaped hose joint 97; an L-shaped third elbow joint 99 attached to the tip of the tube 98 and bent at approximately 90 degrees; and the suction member 58 having a collar and connected to the tip of the third elbow joint 99. Therefore, in this example, the tube 98 is connected to the joint members (the second elbow joint 95 and the T-shaped hose joint 97) fixed to the door 101 and arranged outside the cabinet 100.

On the T-shaped hose joint 97, a cap 97b is detachably attached to the opening end of an intermediate joint 97a which is not connected to the second elbow joint 95 or the tube 98 so that the cap 97b can be removed and liquid can be added through the intermediate joint 97a when it is necessary to replenish the radon extractor 14 with liquid. The joint hose 96 is provided with sufficient extra length so that the first elbow joint 93 and the second elbow joint 95 can stay connected even when the door 101 is opened and closed with the radon generation container 5 being stored in the cabinet 100.

The tube 98, the third elbow joint 99, or the suction member 58 with a collar of the gas channel 15 can be hooked and held by a hanging hook 102 at an appropriate position at the upper part of the door 101.

Furthermore, a gap 103 is formed between the upper end of the door 101 and the upper panel 100a of the cabinet 100, and the tube 98 can be stored in a space 104 formed between the upper surface of the radon generation container 5 and the upper panel 100a of the cabinet 100 via the gap 103.

In the configuration described above, when external air is introduced into the radon reservoir 8 to use the radon inhalation device 1, the stopper 79 attached to the end, of the external air introducing pipe 78, protruding outside the door 101 is removed, and from that state, the air suction machine 110 is inserted to the opening part, of the air suction pipe 85, protruding outside the door 101. Then the air inside the external air opening chamber 10 is suctioned by activating the air suction machine 110.

Then, as the air inside the external air opening chamber 10 is suctioned by the air suction machine 110 through the air suction channel 82 and released outside the radon generation container 5, the pressure inside the external air opening chamber 10 is decreased to be negative. And as the air inside the external air opening chamber 10 decreases, air is introduced inside the radon reservoir 8 via the radon extractor 14 and the external air introducing pipe 78, and the bag 11 is lifted. In the state where the air inside the external air opening chamber 10 is suctioned sufficiently, as shown in FIG. 20A, the bag 11 is in contact with the inner surface of the upper case member 81 in a wide range, and the space inside the radon reservoir 8 is at its maximum (almost 50 liters).

Here, the inside of the case of the elbow joint 83 to which the air suction pipe 85 is connected is bent upward, and the support pipe 86 is extended upward along the inner surface of the side wall 81b. Thus, the bag 11 does not get caught by the elbow joint 83 and the support pipe 86 or block the end opening 86a of the support pipe 86. In addition, as the plurality of through holes 86b are provided on the peripheral wall of the support pipe 86, even when part of the through holes 86b is blocked by the bag 11, the air inside the external air opening chamber 10 can be continuously suctioned via the other through holes or the upper opening.

After external air is introduced into the external air opening chamber 10 as described above, the stopper 79 is attached to the opening end, of the external air introducing pipe 78, protruding from the door 101 to seal the external air introducing channel 72. This prevents the gas containing radon from being leaked from the radon reservoir 8 and prevents the radon concentration inside the radon reservoir 8 from being reduced by introduction of external air into the radon reservoir 8 at the time of inhalation of the gas containing radon in the radon reservoir 8.

In a case where the gas containing radon is inhaled thereafter, the part of the gas channel 15 hooked on the hanging hook 102 is detached and the tube 98 stored in the space 104 between the radon generation container 5 (container main body 4) and the top panel 100a of the cabinet 100 is pulled out to be guided to a desired position. Then, a user put their mouth at the suction member 58 to inhale the gas. This makes it possible to inhale the gas containing radon from the radon reservoir 8 via the radon extractor 14 and the gas channel 15 on the principle similar to that of the former embodiments.

As the gas containing radon reserved in the radon reservoir 8 is continuously inhaled via the gas channel 15, the amount of air inside the radon reservoir 8 is decreased, causing the bag 11 to descend, and external air is flown into the external air opening chamber 10 via the air suction pipe 85.

After the gas containing radon in the radon reservoir 8 is almost completely gone, as shown in FIG. 20B, the bag 11 is pulled down to the bottom of the lower case member 71, and the space inside the radon reservoir 8 is almost gone. In a case where a user wants to inhale the gas containing radon after that, the stopper 79 attached to the opening end of the external air introducing pipe 78 is removed again, and from that state, the air suction machine 110 is inserted to the opening end of the air suction pipe 85, and the air inside the external air opening chamber 10 is suctioned by activating the air suction machine 110, which is a repetition of the operation described previously.

Therefore, in the sixth embodiment described above, the bag 11 is lifted by suction of the air inside the external air opening chamber 10 by the air suction machine 110, which can increase the volume of the radon reservoir 8, and a user need not take out the radon generation container 5 out of the cabinet 100 and open the upper case member 81 to lift the bag 11. As it is unnecessary for the user to access the radon generation container 5, the door 101 may be provided with a lock 120 (shown in FIG. 19B) which keeps the door 101 closed during usual use.

As described above, the gas containing radon is sucked with vapor (water droplets), which reduces the remaining amount of the liquid 34 stored at the lower part of the radon extractor 14. The radon extractor 14 may be replenished with the liquid via the intermediate joint 97a of the T-shaped hose joint 97 provided on the gas channel 15. Even though the liquid may be supplied from anywhere on the gas channel 15, the radon extractor 14 can be replenished easily via the joint hose 96 and the elbow joints 91, 93, 95: as the intermediate joint 97a of the T-shaped hose joint 87 provided on the gas channel 15 outside and near the door 101 is directed upward; the cap 97b attached to the tip of the intermediate joint 97a is removed; and the liquid is added via the intermediate joint 97a of the T-shaped hose joint 97 with a spuit or the like.

A viewing window 105 (shown in FIG. 13B) may be formed in the side wall 71b of the lower case member 71 of the radon generation container 5 at a part facing the radon extractor 14 so that the remaining amount of the liquid inside the radon extractor 14 can be visually seen.

### REFERENCE SIGNS

1 . . . . RADON INHALATION DEVICE
4 . . . . CONTAINER MAIN BODY
5 . . . . RADON GENERATION CONTAINER
6 . . . . HEATER
7 . . . . RADIOACTIVE MATERIAL CONTAINING ORES
8 . . . . RADON RESERVOIR
10 . . . . EXTERNAL AIR OPENING CHAMBER
11 . . . . BAG
14, 62 . . . . RADON EXTRACTORS
15 . . . . GAS CHANNEL
26 . . . . CENTER CHANNEL
27 . . . . SIDE CHANNEL
28 . . . . CONNECTION PORT
39 . . . . INTERNAL SPACE
57, 98 . . . . TUBES
63 . . . . FILTER
71 . . . . LOWER CASE MEMBER
72 . . . . EXTERNAL AIR INTRODUCING CHANNEL
76, 83, 91, 93, 94 . . . . ELBOW JOINTS
78 . . . . EXTERNAL AIR INTRODUCING PIPE
81 . . . . UPPER CASE MEMBER
82 . . . . AIR SUCTION CHANNEL
85 . . . . AIR SUCTION PIPE
86 . . . . SUPPORT PIPE
95 . . . . SECOND ELBOW JOINT
97 . . . . T-SHAPED HOSE JOINT
100 . . . . CABINET
101 . . . . DOOR
110. AIR SUCTION MACHINE
111, 112 . . . . THROUGH HOLES

1. A radon inhalation device comprising: a radon generation container for producing radon; and a heater that heats an inside of the radon generation container,
wherein the radon generation container includes: a container main body an inside of which is divided into a radon reservoir and an external air opening chamber by a bag; a radioactive material containing ore stored inside the radon reservoir; and a radon extractor that communicates with an inside of the radon reservoir and an outside of the container main body,
wherein when the bag is deformed to increase a volume of the radon reservoir, the radon extractor can introduce external air to the radon reservoir, and when gas in an internal space is suctioned from the outside of the container main body, the radon extractor releases radon inside the radon reservoir to the outside of the container main body.

2. The radon inhalation device according to claim 1,
wherein the radon extractor includes parallel channels lower parts of which communicate with each other via a connection port, one of the parallel channels being open to the radon reservoir, another one of the parallel channels being open to the outside of the container main body, and when liquid is contained at the lower part of the parallel channels, a liquid level of the liquid is above the connection port.

3. The radon inhalation device according to claim 1,
wherein the radon extractor includes parallel channels,
wherein the parallel channels include a center channel and a pair of side channels positioned on both sides of the center channel, a lower part of the center channel and a lower part of the pair of the side channels communicating via a connection port, the center channel and one of the side channels being open to the radon reservoir, and the center channel and another one of the side channels being open to the outside of the container main body, and when liquid is contained at the lower part of the parallel channels, a liquid level of the liquid is above the connection port.

4. The radon inhalation device according to claim 1,
wherein a filter to remove floating matter in gas is stored in the radon extractor.

5. The radon inhalation device according claim 1,
wherein the container main body is formed with a lower case member and an upper case member which are fitted to each other in an up-down direction, and the bag is fixed by being sandwiched between the lower case member and the upper case member.

6. The radon inhalation device according to claim 1, further comprising:
a gas channel through which the radon extractor communicates with the outside of the container main body;
an air suction channel that communicates with the external air opening chamber and the outside of the container main body; and
an external air introducing channel through which the radon reservoir and the outside of the container main body communicate with each other and that can be opened and sealed with a stopper,
wherein air in the external air opening chamber can be suctioned by an air suction machine connected to the air suction channel.

7. The radon inhalation device according to claim 6,
wherein the air suction channel includes:
a joint member fixed to the container main body;
an air suction pipe one end of which is connected to the joint member outside the container main body and another end of which can be connected to the air suction machine; and
a support pipe one end of which is connected to the joint member in the external air opening chamber inside the container main body and another end of which is open upward and extended upward.

8. The radon inhalation device according to claim 6,
wherein the external air introducing channel includes:
a joint member fixed to the container main body; and
an external air introducing pipe one end of which is connected to the joint member outside the container main body and another end of which can be opened and sealed with a stopper.

9. The radon inhalation device according to claim 6,
wherein the radon inhalation device is housed in a cabinet with a door,
wherein the gas channel, the air suction channel, and the external air introducing channel are drawn outside via the door.

10. The radon inhalation device according to claim 9,
wherein the air suction channel includes an air suction pipe placed outside the container main body,
wherein the external air introducing channel includes an external air introducing pipe placed outside the container main body,
wherein the gas channel includes a tube placed outside the container main body,
wherein the air suction pipe and the external air introducing pipe are drawn outside the cabinet via a through hole formed in the door, and the tube is connected to a joint member fixed to the door and placed outside the cabinet.
